(19) United States
(12) Patent Application Publication
(10) Pub. No.: US 2024/0374235 A1
(43) Pub. Date: Nov. 14, 2024

(54) INTRAURETHRAL MEDICAL SYSTEMS AND RELATED METHODS

(71) Applicant: Boston Scientific Medical Device Limited, Galway (IE)

(72) Inventors: Nachiket GOLE, Pune (IN); Nitesh Ghananil BAVISKAR, Kalyan West (IN); Rajivkumar SINGH, Thane (IN); Mihir SUKHATME, Thane (IN); Arun ADHIKARATH BALAN, Aluva (IN); Aditya DHANOTIYA, Indore (IN); Pooja Bhuvanesh KULKARNI, Pune (IN)

(73) Assignee: Boston Scientific Medical Device Limited, Galway (IE)

(21) Appl. No.: 18/658,327

(22) Filed: May 8, 2024

Related U.S. Application Data
(60) Provisional application No. 63/500,978, filed on May 9, 2023.

(51) Int. Cl. A61B 8/12; A61B 8/00; A61B 10/04; A61B 90/00

(52) U.S. Cl. CPC A61B 8/12; A61B 8/4488; A61B 8/469; A61B 8/587; A61B 10/04; A61B 2010/045; A61B 2090/3925

(57) ABSTRACT

Medical systems and methods of use thereof useful to image the body are described. The medical system may include a first medical device defining a channel between a proximal end and a distal end of the first medical device; and a second medical device including a tube, a plunger, and a needle. A distal portion of the tube may include an imager, and the needle may be translatable along the tube relative to the plunger. The second medical device may be insertable in, and translatable along, the channel of the first medical device.

### CROSS-REFERENCE TO RELATED APPLICATIONS

This application claims the benefit of priority to U.S. Provisional Application No. 63/500,978, filed on May 9, 2023, which is incorporated by reference herein in its entirety.

### TECHNICAL FIELD

Aspects of this disclosure generally relate to medical systems and procedures. Particular aspects relate to medical systems, devices, and methods for intraurethral analysis and/or diagnosis.

### BACKGROUND

Screening for cancer in the male reproductive system and urinary tract presents various challenges. For example, the relatively small size of the prostate and its location make it difficult to accurately examine the entire organ. The prostate surrounds the urethra, positioned just below the bladder and in front of the rectum. While existing methods examine the prostate through the rectum, this approach is limiting in terms of the ability to accurately examine all tissues. Magnetic resonance imaging is expensive and is not available in many clinical settings, and such imaging techniques fail to provide biopsy samples. Furthermore, it is often difficult to define the location of cancerous or potentially cancerous tissue.

### SUMMARY

The present disclosure includes medical systems and methods of use thereof useful to image the body. For example, the present disclosure includes a medical system comprising a first medical device defining a channel between a proximal end and a distal end of the first medical device; and a second medical device including a tube, a plunger, and a needle, wherein a distal portion of the tube includes an imager, and wherein the needle is translatable along the tube relative to the plunger; wherein the second medical device is insertable in, and translatable along, the channel of the first medical device. In some examples, the first medical device may be referred to as a hub, and the second medical device may be referred to as a probe.

According to some aspects of the present disclosure, the first medical device includes a distal tube surrounding at least a distal portion of the channel, wherein the distal tube is configured to be inserted within a bodily lumen, such as a urethra. An outer surface of at least one of the first medical device or the second medical device may include a marking for alignment of the second medical device relative to the first medical device. The tube of the second medical device may include a radial opening proximate the imager, the needle being extendable through the radial opening. Optionally, the plunger includes a plurality of markings, each marking of the plurality of markings corresponding to a distance the needle is extendable through the radial opening. In some examples, the distance is at least 0.1 cm, for example ranging from about 1 cm to about 10 cm.

The second medical device may be rotatable relative to the first medical device when inserted into the channel so that an orientation of the radial opening relative to the first medical device is adjustable. A proximal end of the plunger may include a base and/or a proximal end of the needle may include a needle hub, e.g., the needle hub configured to abut the base when the needle is fully extended within the tube. In some examples, the needle is pre-formed to bend at an angle, such as an angle between about 60° and about 90°. The imager may be configured to capture a 360-degree view around the distal portion of the tube of the second medical device. According to some aspects of the present disclosure, the imager comprises an ultrasound phased array transducer. The second medical device may include a sensor electronically marked for calibration of the medical system. The medical system may further comprise a processor in communication with the imager and configured to calibrate the medical system. The medical systems described above and elsewhere herein may be used to screen tissue of a subject's urinary system. For example, the imager may be configured to capture a plurality of ultrasound images to examine the subject's urethra, prostate, and/or bladder.

The present disclosure also includes a medical system comprising a first medical device defining a channel between an option of a proximal end and an opening of a distal end of the first medical device; and a second medical device including a tube, a plunger, and a needle, wherein a distal portion of the tube includes an ultrasound imager, wherein the needle is translatable along the tube relative to the plunger, and wherein the tube includes a radial opening, the needle being extendable through the radial opening; wherein the second medical device is insertable in, and translatable along, the channel of the first medical device. In some examples, an outer surface of the second medical device includes a marking for alignment of the second medical device relative to the first medical device. The second medical device may be rotatable relative to the first medical device when inserted into the channel, e.g., so that an orientation of the radial opening relative to the first medical device is adjustable.

The present disclosure also includes a method of treating a subject, the method comprising inserting a first medical device into a bodily orifice or lumen of the subject; inserting a second medical device into a channel of the first medical device and translating the second medical device along the channel until a distal portion of the second medical device is within a body cavity of the subject; capturing at least one image of the body cavity via an imager of the distal portion of the second medical device; analyzing the at least one image to identify an area of interest of the body cavity; and taking a sample of tissue from the area of interest by extending a needle of the second medical device through a radial opening of the second medical device. The bodily orifice or lumen may be a urethra, for example. The method may further comprise calibrating a position and/or orientation of the second medical device relative to the body cavity after capturing the at least one image and before taking the sample of tissue. Analyzing the at least one image may include identifying a distance between a central axis of the second medical device and the area of interest. According to some aspects herein, the needle may be extended the distance through the radial opening, e.g., to take the sample of tissue.

### BRIEF DESCRIPTION OF THE DRAWINGS

The accompanying drawings are incorporated in and constitute a part of this application. The drawings illustrate aspects of the disclosure that, together with the written description herein, serve to explain this disclosure. Each drawing depicts one or more exemplary aspects according to this disclosure, as follows:

FIGS. 1A-1B depict an exemplary medical system, according to aspects of the present disclosure.

FIGS. 2A-2D depict an exemplary method of using the medical system shown in FIGS. 1A-1B.

FIGS. 3A-3B depict exemplary images obtained with the medical system shown in FIGS. 1A-1B.

FIG. 4 is a schematic for a series of images taken at different positions along the urethra and prostate during the exemplary method shown in FIGS. 2A-2D.

### DETAILED DESCRIPTION

In view of the aforementioned challenges, there remains a need for improved systems and devices for examination and/or diagnosis of the prostate and other organs and tissues of the urinary tract and male reproductive system. The present disclosure may facilitate early detection of cancer, identifying cancerous tissue and tissues suspected of having cancer, accessing targeted tissues for biopsy, and/or monitoring prostate health and the health of other tissues post treatment for a duration of time. While aspects of the present disclosure may address one or more limitations of prior devices and methods, the scope of the disclosure, however, is defined by the attached claims and not the ability to solve a specific problem.

Aspects of the present disclosure are described with reference to exemplary systems and methods for imaging and detection of irregularities within a body, e.g., the urinary tract. Irregularities may refer to any abnormalities including growths (benign or malignant), wounds, cancerous tissue, etc. Some aspects are described with reference to medical procedures, e.g., intraurethral procedures, in which a probe is guided through a portion of the body until a distal end of the probe is located in or proximate to a body cavity that is to be imaged. For example, the probe may include an elongated tube or sheath that is guided through a urethra, e.g., via a guiding hub, until a distal end of the tube or sheath is located in a bladder or a prostate. References to a particular type of medical procedure, such as an intraurethral procedure, are provided for convenience and illustrative purposes, and are not intended to limit the present disclosure. Accordingly, the concepts described herein may be utilized for analogous medical devices and methods, which are contemplated herein.

Wherever possible, the same or similar reference numbers will be used throughout the drawings to refer to the same or like parts. The term “distal” refers to a portion farthest away from a user when introducing a device into a subject (e.g., patient). By contrast, the term “proximal” refers to a portion closest to the user when placing the device into the subject.

Both the foregoing general description and the following detailed description are exemplary and explanatory only and are not restrictive of the features as claimed. As used herein, the terms “comprises,” “comprising,” “having,” “including,” and other variations thereof are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms such as, for example, “about,” “substantially,” “generally,” and “approximately” are used to encompass ±10% of a stated value or characteristic.

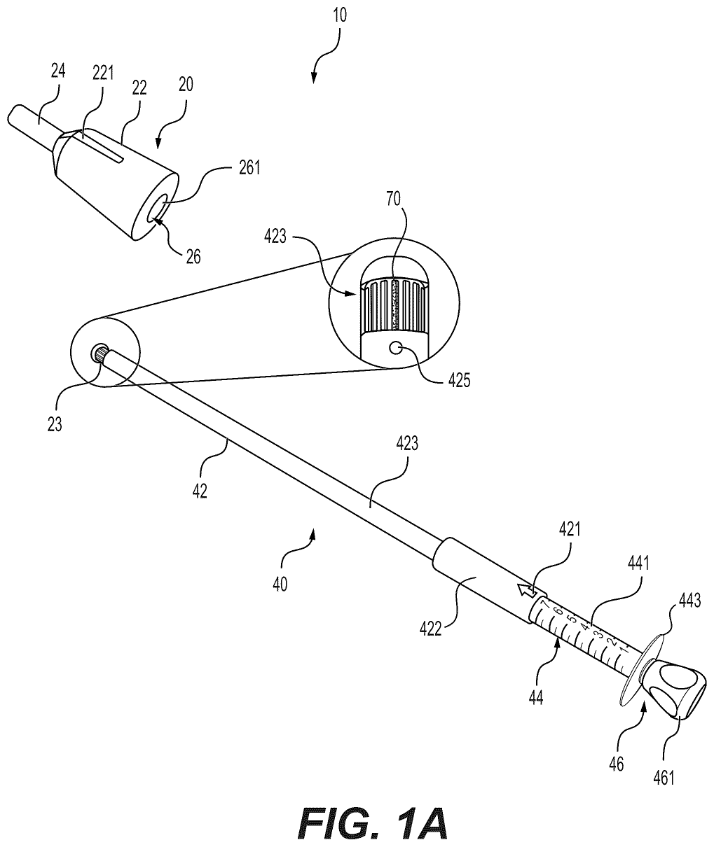

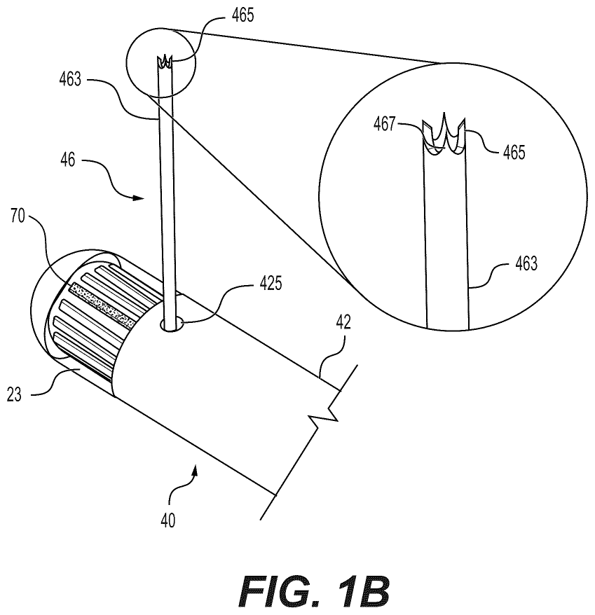

An exemplary medical system 10 according to the present disclosure is shown in FIGS. 1A and 1B. System 10 comprises a first medical device, e.g., a hub 20, and a second medical device, e.g., a probe 40. Hub 20 and probe 40 may be two separate components. Hub 20 may be at least partially insertable within a bodily orifice or lumen, e.g., a urethra, and may include a channel 26 configured to receive probe 40. For example, hub 20 may include a body 22 and a projection such as an elongated tube 24, wherein the body 22 or both the body 22 and the tube 24 define channel 26 therein.

As shown in FIG. 1A, body 22 may be proximal to tube 24, as tube 24 extends distally from body 22. Body 22 may be of any suitable shape, such as, e.g., cylindrical, conical, pyramidal, etc. Body 22 may be of larger cross-sectional dimension relative to tube 24 (e.g., body 22 may have a greater diameter than that of tube 24). Body 22 may include a proximal surface, which may include a proximal opening 261 configured to receive probe 40 within channel 26. The proximal surface of hub 20 may provide a surface against which aspects of probe 40 may abut against to inhibit further insertion of probe 40 within hub 20. Optionally, body 22 may include surface or other structural features such as, e.g., one or more indents, grips, etc., to facilitate handling and/or gripping of hub 20 by a user. According to some aspects of the present disclosure, body 22 may include one or more markings to help guide alignment of probe 40 relative to body 20. For example, as shown in FIG. 1A, an outermost surface of hub 20 may include at least one marking 221, e.g., in the form of an alignment line 221.

The distal projection, e.g., tube 24, may be of any suitable cross-sectional dimension that allows for insertion into a bodily orifice or lumen, e.g., a urethra. The tube 24 may have a size that allows for hub 20 to be securely held within the bodily orifice or lumen. Tube 24 may comprise any suitable biocompatible material(s), e.g., polymer(s), suitable metals or metal alloys such as stainless steel, etc. Optionally, hub 20 may include features to help tube 24 to stay in place within a bodily orifice or lumen. For example, adhesive tape may be used to maintain the position of hub 20 within the patient once inserted. In some aspects, channel 26 of hub 20 may extend between proximal opening 261 of body 22 and a distal opening of tube 24.

As mentioned above, probe 40 may be insertable within hub 20 and guided through a bodily orifice or lumen, e.g., the urethra. Referring to FIGS. 1A and 1B, probe 40 may include a sheath 42, a plunger 44, and a needle 46. Sheath 42 may include a proximal body 422 and a tube 423 that extends distally from body 422. Body 422 may be tubular or cylindrical in structure. In some examples, body 422 may have a greater cross-sectional dimension than tube 423 so that body 422 radially protrudes outwards relative to tube 423. Optionally, body 422 may include one or more markings, e.g., arrow 421 depicted in FIG. 1A, to assist with alignment relative to hub 20. For example, arrow 421 may be aligned with alignment line 221 of hub 20, e.g., for calibration as discussed further below. In some examples, body 422 include one or more features such as protrusions, indents, etc., to assist in the handling and/or gripping of probe 40 by a user. Body 422 may include a proximal opening that receives plunger 44 as shown in FIG. 1A. Tube 423 may have an elongated tubular structure extending distally from the proximal end of body 422.

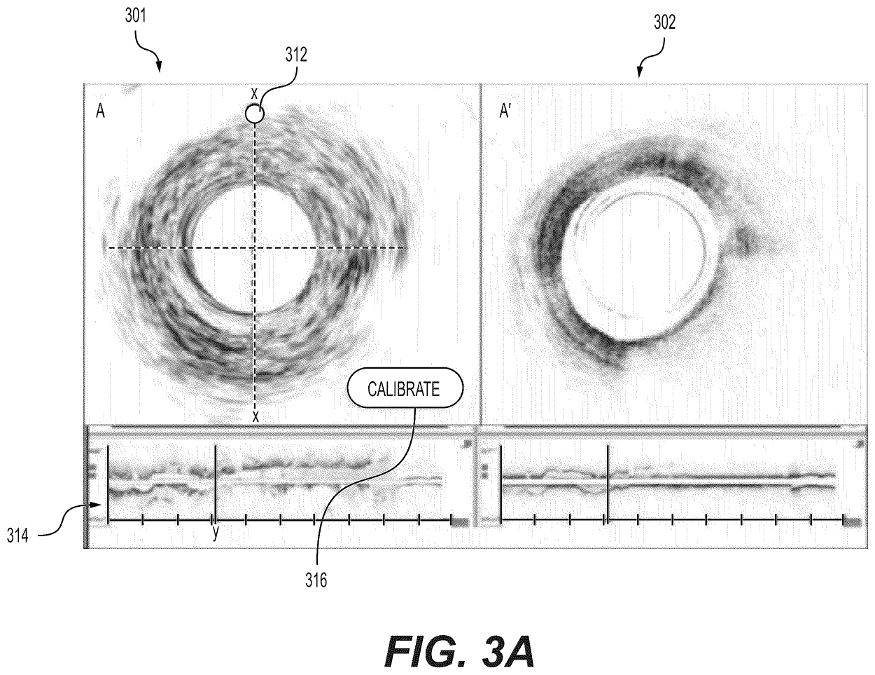

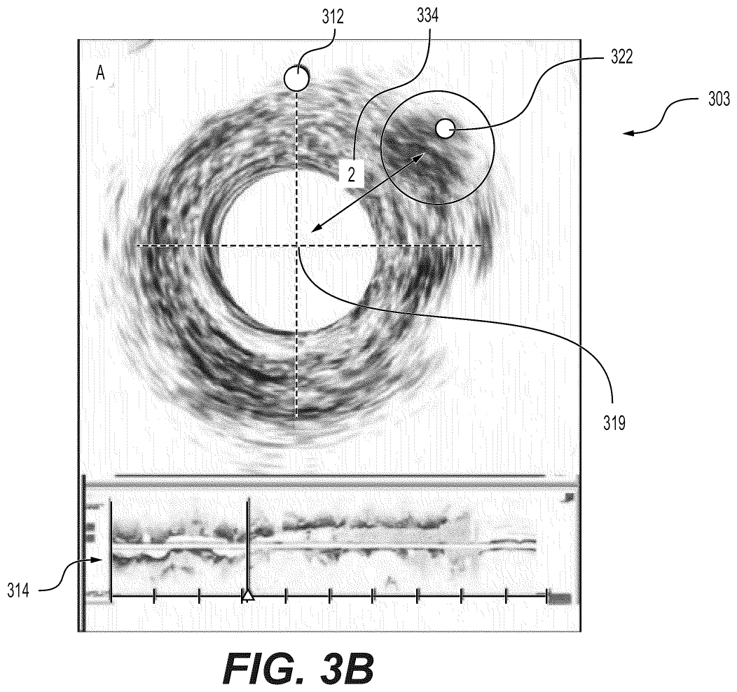

A distal portion or end of tube 423 may include an imager 23 configured to capture images of the surrounding environment. For example, imager 23 may be configured to capture ultrasound images by emitting and receiving sound waves. Imager 23, in some examples, may include an ultrasound transducer, e.g., a phased array ultrasound transducer. In some aspects of the present disclosure, the imager 23 may be configured to capture a 360-degree view of the surrounding tissue, e.g., the imager 23 including a plurality of sensors or transducers in an annular configuration about the longitudinal axis of probe 40. When placed within a body cavity, imager 23 may be used to examine outer boundaries of organs, tissue, etc., proximate probe 40. Thus, for example, imager 23 may be used to screen for the presence of irregularities in an outer profile of organs and/or other tissue of a patient, and to identify targets for biopsy. Imager 23 may be in communication with capital equipment configured to run imaging software, to display the images for a user (e.g., image 301 in FIGS. 3A-3B) and interact with software via said display.

A distal portion or end of probe 40 may include a distal opening 425. Distal opening 425 may be adjacent to or otherwise proximate imager 23. Distal opening 425 may be positioned radially on a surface of body 422, e.g., the distal opening 425 being a radial opening. The dimensions of distal opening 425 may allow for a distal portion of needle 46 to extend and retract through distal opening 425, as shown in FIG. 1B. Sheath 42 may include a channel extending between distal opening 425 and a proximal opening of probe 40 to receive needle 46. In some examples, sheath 42 or another portion of probe 40 may include a sensor useful for calibration of probe 40.

Plunger 44 may assist with taking a biopsy when desired, e.g., controlling extension of needle 46 for taking a tissue sample and retrieving the sample for further study. At least a portion of plunger 44 may extend through the channel of sheath 42. Plunger 44 may be configured to extend and/or retract through the proximal opening of body 422, as plunger 44 translates within the channel of sheath 42. A proximal portion of plunger 44 may include markings to assist a user with determining the position of needle 46. For example, plunger may include at least one marking, e.g., a plurality of markings 441. Markings 441, in some instances, may denote a unit of measurement such as, e.g., millimeters (mm), centimeters (cm), etc. Markings 441 may denote a distance for needle 46 to extend in a direction, e.g., a radial direction, from distal opening 425 once plunger 44 is translated distally within sheath 42 until a proximal end of body 422 aligns with one or markings 441. Thus, for example, translation of plunger 44 until the proximal end of body 422 aligns with a desired distance marking 441 may indicate that needle 46, in a fully extended position, extends a corresponding distance in a radial direction through distal opening 425. A proximal end of plunger 44 may include a base 443. A proximal surface of base 443 may provide an abutment against a distal surface of a needle hub 461, as shown in FIG. 1A.

Still referring to FIGS. 1A and 1B, needle 46 as shown includes needle hub 461 and a needle body 463 including a distal end 465. Needle body 463 may extend distally from a proximal portion or end of needle hub 461. Needle body 463 may comprise a suitable biocompatible material or combination of materials. In some examples, needle body 463 comprises an elastic or otherwise deformable material, e.g., Nitinol, so that needle body 463 may be pre-formed to bend at a specified angle, e.g., an angle ranging from about 60° to about 90°, such as an angle of about 60°, about 75°, or about 90°, etc. For example, as shown in FIG. 1B, a portion of needle body 463 may be pre-formed to adopt a 90° angle so that needle body 463 may extend through distal opening 425 in a radial or perpendicular direction relative to a longitudinal axis of sheath 42. Needle body 463 may be of a suitable width, and may be of a sufficient length to at least extend through distal opening 425, in a radial direction, as far as indicated by each of markings 441. Thus, in some examples, needle body 463 may have a length sufficient to extend the needle 46 by a distance ranging from about 1 cm to about 10 cm through the distal opening 425, a distance ranging from about 1 cm to about 5 cm, or from about 2 cm to about 7 cm. According to some aspects of the present disclosure, distal end 465 of needle body 463 may include a plurality of prongs 467. Each of prongs 467 may include an edge configured to tear and hold tissue, e.g., for biopsy. Edges of prongs 467 may additionally include various features, e.g., serrated aspects, which may assist in tissue retrieval and hold.

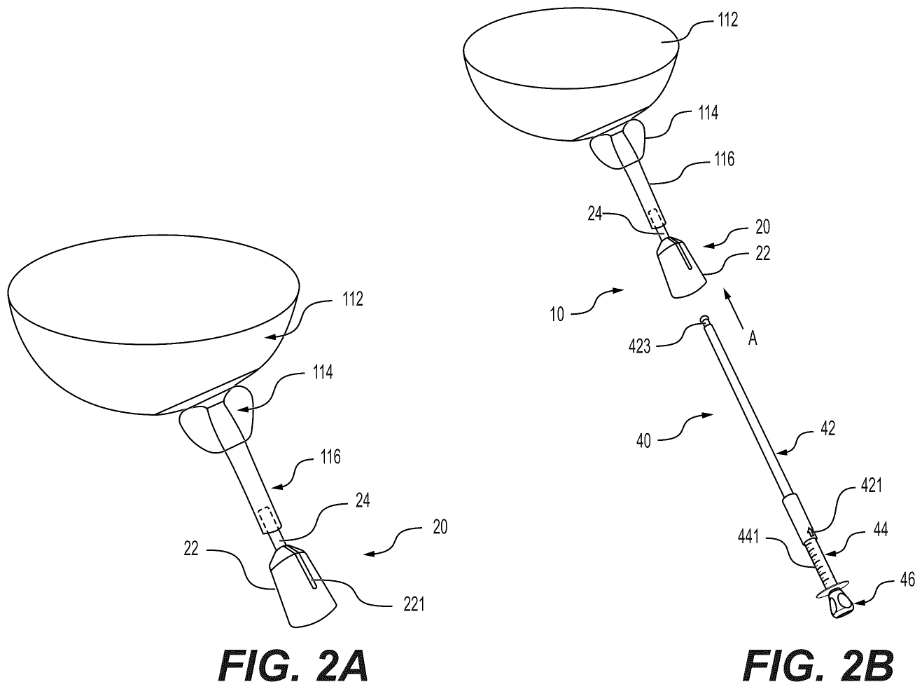

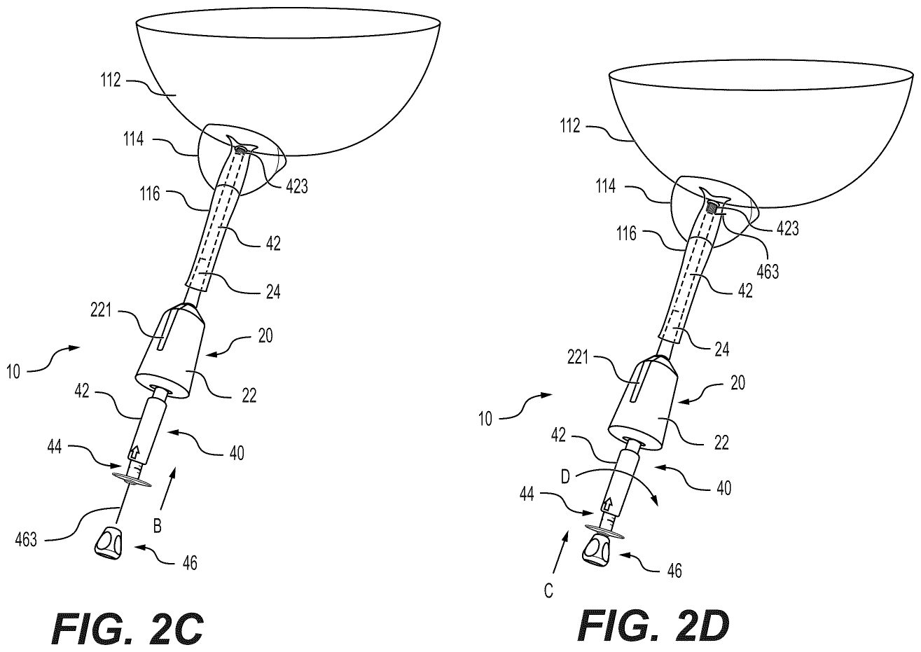

Referring to FIGS. 2A-2D, an exemplary process of using medical system 10 is discussed. In this example and for illustrative purposes, the medical system 10 is used to image and/or biopsy tissue of a subject's urinary tract, wherein the figures show the urethra 116, prostate 114, and bladder 112 (other anatomical features omitted for clarity). As shown in FIG. 2A, elongated tube 24 of hub 20 may be inserted through a bodily opening and within a bodily orifice or lumen, e.g., urethra 116. Hub 20 may be inserted within urethra 116 so that alignment line 221 of body 22 may face a particular direction. For example, hub 20 may be inserted and rotated, if necessary, so that alignment line 221 faces the user (e.g., physician or other medical professional) of the system 10 and the anatomical anterior of the subject. That is, hub 20 may be rotated until alignment line 221 is in the subject's anterior direction. Hub 20 may be maintained in the same position relative to the subject (e.g., referred to as the anterior position of hub 20), e.g., to assist with determining the desired position and orientation of probe 40 relative to hub 20 and relative to various tissues of the subject.

After insertion and placement of hub 20 within urethra 116, probe 40 may be inserted through proximal opening 261 (shown in FIG. 1A) of hub 20, as indicated by directional arrow A in FIG. 2B. During and/or after insertion of probe 40 within channel 26 of hub 20, probe 40 may be rotationally positioned relative to hub 20 so that arrow 421 is aligned with marking(s), e.g., alignment line 221, of hub 20. Probe 40 may be rotatable relative to hub 20 when inserted into channel 26 so that an orientation of distal opening 425 (e.g., radial opening) relative to hub 20 is adjustable. After insertion of probe 40 within hub 20, probe 40 may extend distally through the distal opening of elongated tube 24 and imager 23 used to capture images of the surrounding anatomy, the images being shown on a display.

According to some aspects of the present disclosure, probe 40 may be calibrated before and/or after a procedure. For example, probe 40 may be advanced through urethra 116 and prostate 114 until the imager 23 reaches the bladder 112, as shown in FIG. 2C, such that bladder 112 is visible on the display. Because the outer boundary of bladder 112 is substantially wider than that of prostate 114, this position of imager 23 may be useful for orienting probe 40 relative to the surrounding anatomy.

Exemplary ultrasound images 301, 302 of bladder 112 captured using imager 23 when imager 23 is proximate the bladder 112 (FIG. 2C) are shown in FIG. 3A. In images 301, 302, the circle represents the distal starting point of urethra 116 and the starting point to image prostate 114. Because imager 23 in this example is configured as an annular array of transducers, the images 301, 302 provide a 360-degree view of the tissue surrounding imager 23. At this point, the user may calibrate the probe 40, e.g., using software (e.g., a processor in communication with imager 23), using a sensor 70 on the anterior side of probe 40 (the side aligned with alignment line 221 of hub 20) that is electronically marked for calibration. Sensor 70 may be one of a plurality of sensors of imager 23, that is, the sensor aligned with arrow 421. In some examples, the display includes a touchscreen capable of receiving user input. At this point, the user may calibrate system 10 so that the image 301 on the display rotates and aligns the anterior direction in the image 301 coming from the anterior sensor 70 to correspond to a set reference marker in the crosshairs. For example, as shown in FIG. 3A, the display may show a calibration toggle or button 316, which may be pressed after alignment between alignment line 221 of hub 20 and arrow 421 of probe 40, thereby calibrating probe 40 to an anterior position in accordance with the anterior position of hub 20. Other displays may receive user instructions to calibrate via input other than touchscreen (e.g., via a mouse, keyboard, etc.). Below each image 301, 302 is a depiction 314 of the progression of probe 40 along urethra 116 (left to right corresponding to translation of probe 40 along urethra 116 in a direction from bladder 112 towards prostate 114) as a compilation of images along axis X-X, wherein the vertical line Y marks the current position of imager 23. Marker 312 in images 301, 302 identifies the anterior position of probe 40 in accordance with the calibration sensor.

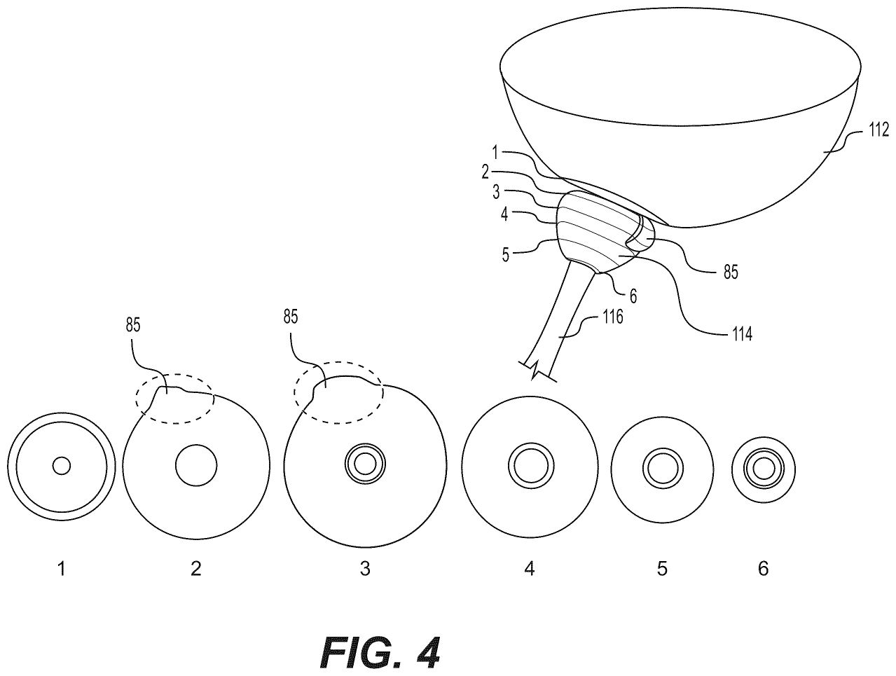

Once probe 40 is calibrated, probe 40 may be translated along urethra 116 to collect a plurality of images that collectively assist the user with screening for tissue abnormalities. Referring to FIG. 3B, the user (and/or the imaging software) may identify an area of interest 322 (e.g., suspicious tissue, possible lesion, etc.) in an image 303 for further examination. In this example, the area of interest 322 is a portion of prostate 114 proximate bladder 112 (see also FIG. 4). The user, using functions of the imaging software, may trace the distance from the center 319 of the image 303 (corresponding to a point along the central axis of probe 40) to area of interest 322. Upon receiving this input, the image 303 may display the distance 334 between center 319 and area of interest 322 (e.g., 2 cm). This distance 334 may assist the user with proper deployment of needle 46 to take a biopsy.

For example, the user may use markings 441 on plunger 44 to determine the distance that needle 46 should be extended in order to contact tissue at the area of interest 322. The user may extend plunger 44 distally relative to sheath 42, as indicated by directional arrow B in FIG. 2C, until the proximal end of sheath 42 aligns with the desired markings 441 that corresponds to distance 334. Thus, for example, the user may translate plunger 44 until the proximal end of body 422 aligns with a “2 cm” marking 441, corresponding to the distance 334 between the central axis of probe 40 and area of interest 322. The user then may extend needle 46, as indicated by directional arrow C in FIG. 2D, until needle hub 461 abuts against proximal surface of base 443 of plunger 44. As a result, needle 46 may extend through distal opening 425 (FIG. 1B) of sheath 42 and may reach area of interest 322, so that distal end 465 of needle 46 may obtain a sample of tissue from area of interest 322. Needle 46 may also be rotated within plunger 44 so that the needle 46 has the appropriate orientation and to assist with tissue retrieval from area of interest 322.

FIG. 4 illustrates how a user may compare (e.g., analyze) multiple images taken with probe 40 in different positions relative to surrounding organs and/or other tissue to screen for abnormalities. Positions marked 1-6 along prostate 114 and urethra 116 correspond to cross-sectional images in FIG. 4 showing variations in the outer boundary of prostate 114 and urethra 116. In cross-sections corresponding to positions 2 and 3, a user may identify an irregular bump 85 localized to the region where prostate 114 meets bladder 112. In position 1 closer to bladder 112, and positions 4, 5, and 6 farther from bladder 112, the outer boundary of prostate 114 is more uniform as expected for healthy tissue. The user therefore may be able to identify the specific area of bump 85 for further examination and to take a tissue biopsy as discussed above.

While principles of the disclosure are described herein with reference to illustrative aspects for particular applications, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

1. A medical system comprising:
a first medical device defining a channel between a proximal end and a distal end of the first medical device; and
a second medical device including a tube, a plunger, and a needle, wherein a distal portion of the tube includes an imager, and wherein the needle is translatable along the tube relative to the plunger;
wherein the second medical device is insertable in, and translatable along, the channel of the first medical device.

2. The medical system of claim 1, wherein the first medical device includes a distal tube surrounding at least a distal portion of the channel, and wherein the distal tube is configured to be inserted within a urethra.

3. The medical system of claim 1, wherein an outer surface at least one of the first medical device or the second medical device includes a marking for alignment of the second medical device relative to the first medical device.

4. The medical system of claim 1, wherein the tube of the second medical device includes a radial opening proximate the imager, the needle being extendable through the radial opening.

5. The medical system of claim 4, wherein the plunger includes a plurality of markings, each marking of the plurality of markings corresponding to a distance the needle extends through the radial opening.

6. The medical system of claim 5, wherein the second medical device is rotatable relative to the first medical device when inserted into the channel so that an orientation of the radial opening relative to the first medical device is adjustable.

7. The medical system of claim 1, wherein a proximal end of the plunger includes a base and a proximal end of the needle includes a needle hub, the needle hub configured to abut the base when the needle is fully extended within the tube.

8. The medical system of claim 1, wherein the needle is pre-formed to bend at an angle.

9. The medical system of claim 8, wherein the angle is between about 60° and about 90°.

10. The medical system of claim 1, wherein the imager is configured to capture a 360-degree view around the distal portion of the tube of the second medical device.

11. The medical system of claim 1, wherein the imager comprises an ultrasound phased array transducer.

12. The medical system of claim 1, wherein the second medical device includes a sensor electronically marked for calibration of the medical system.

13. A medical system comprising:
a first medical device defining a channel between an option of a proximal end and an opening of a distal end of the first medical device; and
a second medical device including a tube, a plunger, and a needle, wherein a distal portion of the tube includes an ultrasound imager, wherein the needle is translatable along the tube relative to the plunger, and wherein the tube includes a radial opening, the needle being extendable through the radial opening;
wherein the second medical device is insertable in, and translatable along, the channel of the first medical device.

14. The medical system of claim 13, wherein an outer surface of the second medical device includes a marking for alignment of the second medical device relative to the first medical device.

15. The medical system of claim 13, wherein the second medical device is rotatable relative to the first medical device when inserted into the channel so that an orientation of the radial opening relative to the first medical device is adjustable.

16. A method of treating a subject, the method comprising:
inserting a first medical device into a bodily orifice or lumen of the subject;
inserting a second medical device into a channel of the first medical device and translating the second medical device along the channel until a distal portion of the second medical device is within a body cavity of the subject;
capturing at least one image of the body cavity via an imager of the distal portion of the second medical device;
analyzing the at least one image to identify an area of interest of the body cavity; and
taking a sample of tissue from the area of interest by extending a needle of the second medical device through a radial opening of the second medical device.

17. The method of claim 16, wherein the bodily orifice or lumen is a urethra.

18. The method of claim 16, further comprising calibrating a position and/or orientation of the second medical device relative to the body cavity after capturing the at least one image and before taking the sample of tissue.

19. The method of claim 16, wherein analyzing the at least one image includes identifying a distance between a central axis of the second medical device and the area of interest.

20. The method of claim 19, wherein the needle is extended the distance through the radial opening.
